(19) United States
(12) Patent Application Publication
(10) Pub. No.: US 2016/0135878 A1
(43) Pub. Date: May 19, 2016

(54) SYSTEM AND METHOD FOR NERVOUS SYSTEM MODULATION

(71) Applicant: GENERAL ELECTRIC COMPANY, SCHENECTADY, NY (US)

(72) Inventors: ADRIAN WARNER, DELAFIELD, WI (US); CLAUDIO PATRICIO MEJIA, CHICAGO, IL (US); DANIEL RICHARD SCHNEIDEWEND, MENOMONEE FALLS, WI (US); RODGER SCHMIT, WEST BEND, WI (US); TIMOTHY STIEMKE, WEST BEND, WI (US); HANS-PETER STOLL, WHITE PLAINS, NY (US); JASMINA BROOKS, DIAMOND BAR, CA (US)

(73) Assignee: GENERAL ELECTRIC COMPANY, SCHENECTADY, NY (US)

(21) Appl. No.: 14/541,673

(22) Filed: Nov. 14, 2014

(51) Int. Cl. A61B 18/14; A61B 18/02; A61B 18/20; A61B 18/12; A61B 19/00

(52) U.S. Cl. CPC A61B 18/1492; A61B 18/1206; A61B 19/5244; A61B 19/54; A61B 18/20; A61B 18/02; A61B 2018/00434

(57) ABSTRACT

A method for nervous system modulation includes operatively connecting an ECG cable having a plurality of surface electrodes to a body of a patient, introducing a catheter having a plurality of catheter electrodes into a blood vessel of the patient, probing a target location within the patient with the catheter to identify nerve tissue with maximum signal propagation in real-time, and reducing signal propagation in the identified nerve tissue.

### BACKGROUND

1. Technical Field

Embodiments of the invention relate generally to nervous system modulation and, more specifically, to a system and method for visualizing, targeting and ablating the nervous system.

2. Discussion of Art

Tissue ablation is the destruction of tissue, typically pathologic tissue, with the aim to cure a disease. Ablation utilizing RF electrode catheters has been used in numerous applications for many years. For example, cardiac ablation is one form of treatment for restoring normal conduction in patients with cardiac arrhythmias. During cardiac ablation, an RF electrode catheter is inserted into a major vein or artery and then guided into the heart of a patient where the sources of aberrant pathways are located, and the aberrant tissue is ablated.

In recent years, the use of ablation therapy has been expanded to treat other diseases and conditions, such as hypertension. In particular, it has been discovered that sympathetic renal activity connected with congestive heart failure may cause unwanted symptoms such as fluid retention and hypertension. Interrupting this sympathetic nerve activity has been found to potentially mitigate these symptoms. One technique for interrupting the renal sympathetic nerve activity is to ablate the sympathetic nerves disposed around the renal arteries utilizing an RF electrode catheter similar to that used in treating cardiac arrhythmias. In particular, typical renal nerve ablation therapies involve the introduction of an ablation catheter into the renal arteries and ablating the arteries at various longitudinal and radial locations along the arteries to disrupting or deactivating the renal nerves, thereby reducing sympathetic nerve drive. This technique is referred to neuromodulation or denervation and has been shown to have potential in achieving a reduction in blood pressure.

As will be readily appreciated, however, neuromodulation or denervation within the renal artery is often carried out without sufficiently identifying the specific location of the nerve tissue or identifying the nerve tissue prior to ablation, resulting in insufficient diagnosis. Indeed, existing methods may be overly-automated and may discount the need to thoroughly understand the local conduction system prior to performing an ablation. In particular, such automated approaches work on the assumption of specific burn pattern types to disrupt the conduction pathway without actually identifying the nerve tissue at issue, and without analyzing the conduction in real-time. Accordingly, existing methods have not been entirely successfully in treating or alleviating the underlying conditions or symptoms to the degree expected, and clinical trials have shown mixed results.

In view of the above, it is desirable to provide a system and method for nervous system modulation that facilitates the exploration, location, diagnosis and treatment of various nervous system disorders and/or conditions, with a minimum of automation, and which are capable of general purpose application.

### BRIEF DESCRIPTION

In an embodiment, a method for nervous system modulation is provided. The method includes the steps of operatively connecting an ECG cable having a plurality of surface electrodes to a body of a patient, introducing a catheter having a plurality of catheter electrodes into a blood vessel of the patient, probing a target location within the patient with the catheter to identify nerve tissue with maximum signal propagation in real-time, and reducing signal propagation in the identified nerve tissue.

In an embodiment, a method of modulating the nervous system of a patient is provided. The method includes the steps of identifying a target location within the patient for exploration, navigating a catheter to the target location, detecting signal propagation within the nervous system at the target location, the signal propagation being indicative of nerve activity, locating nerve tissue at the target location with maximum signal propagation, ablating the nerve tissue with radiofrequency energy to reduce the signal propagation in the nerve tissue, and, during the step of ablating, monitoring the signal propagation in the nerve tissue.

In an embodiment, a system for nervous system modulation is provided. The system includes a user interface having at least one display associated therewith and a patient interface unit operatively connected to the user interface. The patient interface unit is configured to receive electrical signals from electrodes of a catheter positioned at a nerve site within the body of a patient and surface electrodes of an ECG cable attached to the body of the patient, and to provide a digital output corresponding to the electrical signals to the user interface. The user interface is configured to display the electrical signals on the at least one display in real-time and to control the delivery of therapy to the nerve site to reduce signal propagation at the nerve site in dependence upon the real-time display of the signals.

### DRAWINGS

The present invention will be better understood from reading the following description of non-limiting embodiments, with reference to the attached drawings, wherein below:

FIG. 1 is a schematic illustration of a system for nervous system modulation in accordance with an embodiment of the present invention.

FIG. 2 is a schematic illustration of a patient interface unit of the system of FIG. 1.

FIG. 3 is a flowchart illustrating a method of modulating the nervous system of a patient in accordance with an embodiment of the present invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts. Although embodiments of the present invention are described as intended for renal denervation, it will be appreciated that embodiments may be adapted for use in connection with denervation of the nervous system, more generally. As used herein, “electrical contact,” “electrical communication” and “electrically coupled” means that the referenced elements are directly or indirectly connected such that an electrical current may flow from one to the other. The connection may include a direct conductive connection (i.e., without an intervening capacitive, inductive or active element), an inductive connection, a capacitive connection, and/or any other suitable electrical connection. Intervening components may be present.

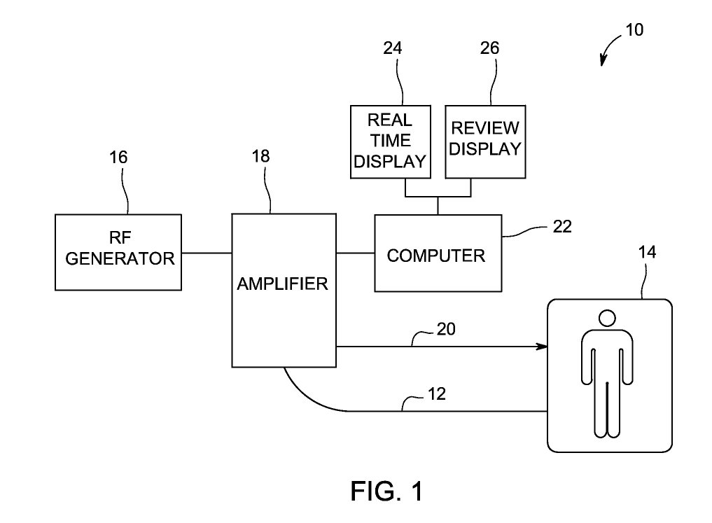

Referring now to FIG. 1, an embodiment of a system 10 for nervous system modulation is depicted. As shown, the system 10 includes a catheter 12 configured to be positioned within the body of a patient 14 and, more particularly, within an artery of a patient 14. In an embodiment, the catheter 12 is configured to be introduced into the patient 14 through the femoral vein or artery in the patient's groin. The catheter 12 is connected to an RF ablation energy source for receiving RF energy, such as an RF generator 16, through a patient interface unit 18. The system also includes an ECG cable 20 having a plurality of surface electrodes that are configured to be attached to a patient 14 and provide a reference to the patient 14 to allow for observation of cardiac function. Both the ECG cable 20 and the catheter 12 are electrically connected to a user interface and control unit 22, such as a computer, through the patient interface unit 18. The user interface and control unit 22 may include, or be associated with, a real-time display 24 and a review display 26, the purposes of which are described hereinafter. As discussed in detail below, the patient interface unit 18 receives electrical signals from the catheter 12 and from the body surface electrodes of the ECG cable 20 and displays the signals on the displays 24, 26. In certain embodiments, the interface 22 may be a Graphical User Interface, such as a computer monitor and keyboard, touch screen, or other human computer interface.

In an embodiment, the catheter 12 may be of any type commonly known in the art and typically includes an elongate body formed of an electrically insulating material, and having an ablation electrode (not shown) at its distal end. A plurality of ECG electrodes (not shown) are provided on the outer surface of the body. In an embodiment, the catheter 12 may be any catheter having a 1:1 electrical connection from the electrodes to the input to the patient interface unit 18. Similarly, the ECG cable 20 may be a standard, 12-lead ECG cable having a plurality of surface electrodes for attaching to the body of a patient.

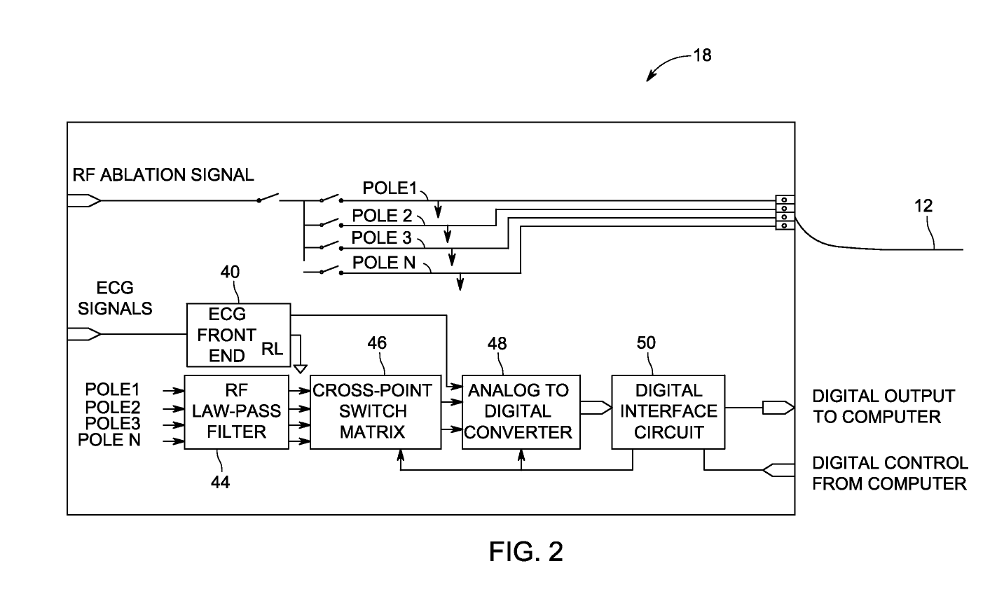

With reference to FIG. 2, a detail, schematic view of the patent interface unit 18 is illustrated. As shown therein, the patient interface unit 18 provides an electrical pathway between the ECG cable 20 and the user interface and control unit 22, enabling ECG signals from the surface electrodes attached to the patient 14 to be received by the user interface and control unit 22 and displayed in usable form on the displays 24, 26. Likewise, ECG signals from the electrodes of the catheter 12 may be transmitted to the user interface 22 though the patient interface 18, and ultimately to displays 24, 26. As will be readily appreciated, this allows for a physician or operator to visualize the conduction of the nervous system at a target location, as discussed in detail below.

In addition, the patient interface unit 18 provides an electrical pathway from the RF generator 16 to the catheter 12, enabling the RF generator 16 to provide RF ablation energy to the patient 14. Moreover, the patient interface unit 18 further provides an electrical pathway between the RF generator 16 and the user interface and control unit 22, enabling a user or operator to control the amount of RF ablation energy provided by the RF generator 16 to the ablation electrode of the catheter 12 during an ablation procedure.

In connection with the above, ECG signals captured by the surface electrodes on the leads of the ECG cable 20 are provided to the patient interface unit 18 through the ECG cable 20 and are received at an ECG front end 40, as is known in the art. In addition to the ECG signals from the ECG surface electrodes of the ECG cable 20, ECG signals captured by the ECG electrodes of the catheter 12 are provided to the patient interface unit 18 and are received by an array of RF low-pass filters 44 provided in an input stage of the patient interface unit 18. These filters 44 are configured to allow an operator of the system 10 to see changes in conduction before, during and after delivery of energy to the ablation electrode of the catheter 12. In particular, these filters 44 prevent interference produced by the application of ablation energy from masking the underlying electrogram wave forms, thereby providing the ability for a user to monitor the electrogram derived from the catheter electrodes to which ablation energy is applied. As will be readily appreciated, therefore, the filters 44 suppress the interference to the electrogram wave forms caused by the application of RF ablation energy provided to the patient via the catheter 12.

The patient interface unit 18 also includes a cross-point switch matrix 46 in communication with the filters 44, which allows for ablation signal steering, as also discussed hereinafter. As shown in FIG. 2, the ECG signals from both the ECG cable 20 and the catheter 12 (once they are received by the filters 44 and cross-switching matrix 46) are provided to an analog-to-digital converter 48. The converter 48 converts the analog signals to digital form and relays the signals to a digital interface circuit 50. In particular, the converter 48 converts the input analog signals from the low-pass filter 44 to a digital value proportional to the magnitude of the voltage or current detected. The interface circuit 50 is configured to provide a digital output to the user interface and control unit 22, allowing a physician to assess and visualize the conduction of the nervous system at the target location, and to receive a digital control signal from the user interface and control unit 22, allowing an operator to steer the catheter to the target location and explore the nervous system at the target location, as well as initiate a burn at the target location to modulate the nervous system. In an embodiment, the patient interface unit 18 is a high-gain amplifier (e.g., 10,000 gain).

As indicated above, the system 10 of the present invention may be utilized for modulating the nervous system, i.e., for disrupting or deactivating nerves that have been identified as causing or contributing to undesirable diseases, conditions or symptoms. For example, the system 10 may be utilized for renal denervation, to treat such conditions as hypertension. In operation, a physician attaches the surface electrodes of the ECG cable 20 to the body of the patient 14 in a manner known in the art, and introduces the catheter 12 into the body of the patient 14, such as through the femoral artery, as is commonly known in the art. As will be readily appreciated, therefore, the patient interface unit 18 is thereby placed in electrical communication with the patient 14 and is capable of receiving ECG signals from the surface electrodes of the ECG cable 20, as well as from the electrodes on the catheter 12 within the patient 14, as discussed in detail above.

In an embodiment, the catheter 14 may be navigated to a specific nerve site identified as requiring therapy using conventional fluoroscopy techniques and systems, and electrical exploratory mapping. In an embodiment, the system 10 may be utilized with the EP Vision 2.0 X-ray mapping system by General Electric Company to assist in steering the catheter to the targeted nerve site. As used herein, “steering” means the process by which a catheter is navigated to a targeted site within the body of a patient.

Once at the nerve site, the electrodes on the catheter 12 are utilized to monitor the conductive activity of the nerves referenced to the patient's heart. In particular, electrodes of the catheter 12, when positioned against tissue in proximity of the nerve ganglion, can detect a signal relative to the patient 14. The electrodes transmit the detected signals to the patient interface unit 18, and to the user interface and control unit 22, where they are displayed in real-time on the real-time display 24. In this manner, a physician viewing the display 24 is able to see the conductive activity of a specific nerve site in real-time. Accordingly, the system 10 of the present invention allows the physician to locate a region of interest, and to then probe the tissue at the region until the area of maximum signal propagation and/or maximum electrical activity can be detected and thus the target nerve site/nerves located. As used herein, “real-time” means substantially real-time and includes any inherent delays in signal transfer from the electrodes to the display 24, such that a physician can visualize electrical signal activity substantially as it is happening, and administer treatment, as discussed below, without exiting the body of the patient.

Once the precise, target nerve site is located, the physician may utilize the user interface and control unit 22 to control the ablation electrode of the catheter 12 to burn the tissue at the target location. In particular, under control of the physician, power at radio frequency is applied to the target location from the ablation power generator 16. Indeed, in an embodiment, ablation energy may be switched from the generator 16, and controlled or steered to the electrode recording sites by the physician so as to modulate or restrict conduction pathways at the target location. In an embodiment, complete destruction of the nerve is possible, but may not be required for certain treatment protocols. As will be readily appreciated, ablation attenuates the electrical pathways within the nervous system and reduces signal propagation.

During ablation, the patient's ECGs are monitored from the surface electrodes of the ECG cable 20 and the electrodes of the catheter 12 and are again displayed on the real-time display 24. Simultaneously, the real-time display 24 may be utilized by a physician to monitor the ongoing conduction activity at the target location as changes of this parameter indicate progression of the ablation lesion. Digitized data is also recorded and saved on the computer 22. Waveform data, ablation energy and duration data, X-ray mapping data can be stored in non-volatile memory within the computer 22 and displayed at later times on the Review Display 26. The ability of a physician to view the change in conduction before, during and after ablation is facilitated by the low-pass filters 44 at the input stage of the patient interface unit 18. Moreover, the ability to measure the conduction at the target location before, during and after ablation allows the physician a ready means to control and direct the treatment. In particular, the system 10 of the present invention provides a physician with the ability to select, energize and monitor the delivery of any RF ablation therapy to an extent heretofore unknown in the art.

As discussed above, the user interface and control unit 22 of the system 10 provides a means for control, display and visualization of the nerve signals at the target location. Indeed, using the standard cardiac reference provided by the ECG cable 20, the patient interface unit 18 to capture the signals, and the switching matrix 46 to enable ablation signal steering, a physician, through the user interface and control unit 22, may then select between a diagnostic signal viewing mode or a therapeutic delivery mode. As discussed, a physician may switch the catheter electrodes to deliver energy to the electrodes to burn the tissue at the target location and thus prevent conduction. Utilizing this technique only requires low energy, typically on the order of 1 Watt but less than 20 Watts.

As will be readily appreciated, the system and method of nervous system modulation of the present invention builds on a study of signals first and provides a method to pinpoint and select ablation site burns, all while utilizing available ablation methods and devices. Thus, the system and method of the present invention involves exploring, locating, diagnosing and treating a target location, with a minimum of automation. Indeed, the system and method of the present invention allows for the exploration of the conduction of the nervous system, which provides the basis for a controlled placement of therapeutic delivery, as well as for the monitoring of the result of the treatment to verify that it was successful. As a result, user control is increased to a degree that has simply not been possible with existing systems and methods. By understanding the conduction and physiological propagation of the nervous system before, during and after treatment, a less aggressive treatment or burn strategy may be implemented, which can help to avoid issues of uncontrolled nerve growth that a ‘blind’ denervation approach may initiate.

Indeed, the ability to probe tissue at a target region to pinpoint the area of maximum signal propagation and/or maximum electrical activity, and then to burn the pinpointed area to achieve denervation while continuously monitoring the electrical activity has simply not been possible with existing methods of renal denervation, which have typically relied on automated procedures and specific burn pattern types, and which are incapable of visualizing or exploring the actual conductive activity within the renal nerves. Moreover, as a result of being automated and incapable of manual exploration at a target site, existing systems are not readily adaptable for use at other nerve sites. Accordingly, existing systems and methods are typically limited to use for renal denervation specifically, and are not applicable to nerve modulation at other nerve sites within the nervous system, more generally. In contrast, the system and method of the present invention provides a means to visualize the conduction of the nervous system for diagnostic determination, exploration and treatment of various disease states such as arrhythmias and other conditions, in addition to high blood pressure via renal denervation.

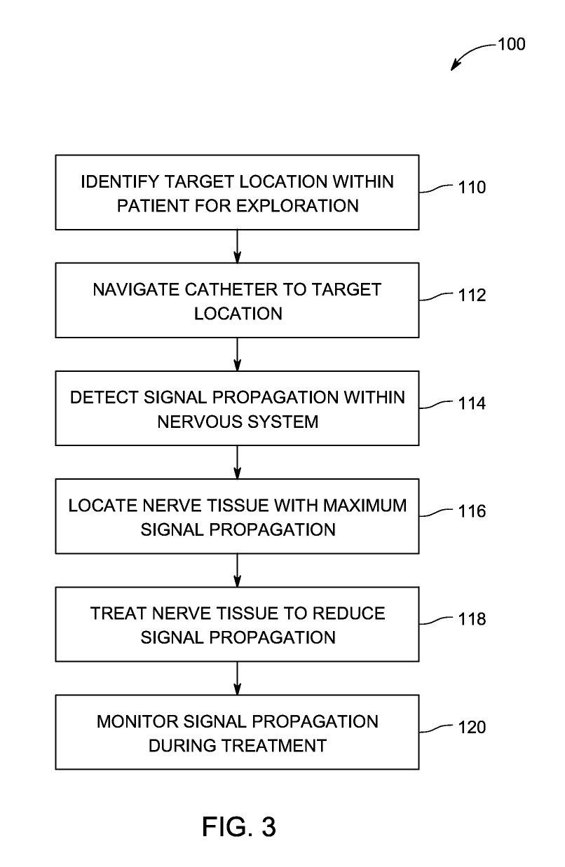

An exemplary method 100 of carrying out the present invention is illustrated in FIG. 3. As shown therein, at step 110, a target location within a patient is identified for exploration. As indicated above, the target location may be a renal artery of a patient having high blood pressure. At step 112, a catheter is navigated to the target location by introducing the catheter into a blood vessel and steering it to the target location. Once at the target location, signal propagation is detected utilizing the electrodes on the catheter, at step 114, until nerve tissue with maximum signal propagation is located, at step 116. After locating the nerve tissue has been located, the nerve tissue may be ablated or treated with a therapy to reduce the signal propagation in the nerve tissue, at step 118. As discussed above, during ablation, the signal propagation in the nerve tissue is monitored in real-time, at step 120 to determine whether the therapy has been successful for reducing the signal propagation in the nerve tissue.

As will be readily appreciated, the system 10 of the present invention is not limited to use with a particular type of catheter. In particular, due to the low energy delivery required by the system, special ablation catheters may not be necessary.

In other embodiments, the system 10 of the present invention may not be restricted solely to RF ablation as a means for modulating or restricting conduction pathways of the nervous system. In an embodiment, it is contemplated that the system 10 of the present invention may be utilized with other forms of therapy delivery where either different methods of intervention are preferred or where an exploratory numbing-type approach is desired or necessary. Such other forms of therapy other than RF ablation may include, but are not limited to, cryoablation, laser ablation or the utilization of agents such as biotoxins. In certain such embodiments, the provided control may be through digital interfaces where a computer or user interface is utilized to control external devices where no electrical signal is delivered to the target therapy site to capture and record key procedural actions.

In an embodiment, a method for nervous system modulation is provided. The method includes the steps of operatively connecting an ECG cable having a plurality of surface electrodes to a body of a patient, introducing a catheter having a plurality of catheter electrodes into a blood vessel of the patient, probing a target location within the patient with the catheter to identify nerve tissue with maximum signal propagation in real-time, and reducing signal propagation in the identified nerve tissue. In an embodiment, the step of reducing signal propagation in the identified nerve tissue may include ablating the identified nerve tissue via an ablation electrode of the catheter. In an embodiment, the ablation electrode is connected to a radiofrequency energy source. In an embodiment, the method may also include the step of monitoring the signal propagation in the nerve tissue while ablating the identified nerve tissue. In an embodiment, the method may include the step of monitoring the signal propagation in the nerve tissue at the target location after ablating the identified nerve tissue, and comparing the signal propagation in the nerve tissue after ablating the identified nerve tissue with the signal propagation in the identified nerve tissue before ablating the identified nerve tissue to determine if the modulation was successful. In an embodiment, the method may include the step of steering the catheter to the target location utilizing at least one of fluoroscopy and electrical exploratory mapping. In an embodiment, the step of probing the target location to identify the nerve tissue with maximum signal propagation includes, at a patient interface unit, receiving electrical signals from the catheter electrodes of the catheter and the surface electrodes of the ECG cable and, at a user interface, displaying the signals on a display. In an embodiment, the display includes a real-time display and a review display. In an embodiment, ablating the identified nerve tissue includes, via the user interface, controlling an output signal from the radiofrequency energy source to the ablation electrode of the catheter. In an embodiment, the target location is within a renal artery of the patient and the identified nerve tissue includes at least one renal nerve.

In an embodiment, a method of modulating the nervous system of a patient is provided. The method includes the steps of identifying a target location within the patient for exploration, navigating a catheter to the target location, detecting signal propagation within the nervous system at the target location, the signal propagation being indicative of nerve activity, locating nerve tissue at the target location with maximum signal propagation, ablating the nerve tissue with radiofrequency energy to reduce the signal propagation in the nerve tissue, and, during the step of ablating, monitoring the signal propagation in the nerve tissue in real-time. In an embodiment, the method may also include the steps of storing signal propagation data representing the signal propagation in the nerve tissue prior to ablation, and monitoring the signal propagation in the nerve tissue after ablating the nerve tissue. In an embodiment, the method may include the step of comparing the signal propagation in the nerve tissue after ablating the nerve tissue with the stored signal propagation data to determine if the ablation was successful. In an embodiment, the step of navigating the catheter to the target location includes steering the catheter utilizing at least one of fluoroscopy and electrical exploratory mapping. In an embodiment, ablating the nerve tissue includes controlling the amount of the radiofrequency energy provided to an ablation electrode on a distal tip of the catheter. In an embodiment, the target location may within a renal artery of the patient and the nerve tissue may include at least one renal nerve.

In an embodiment, a system for nervous system modulation is provided. The system includes a user interface having at least one display associated therewith and a patient interface unit operatively connected to the user interface. The patient interface unit is configured to receive electrical signals from electrodes of a catheter positioned at a nerve site within the body of a patient and surface electrodes of an ECG cable attached to the body of the patient, and to provide a digital output corresponding to the electrical signals to the user interface. The user interface is configured to display the electrical signals on the at least one display in real-time and to control the delivery of therapy to the nerve site to reduce signal propagation at the nerve site in dependence upon the real-time display of the signals. In an embodiment, the system includes an ablation RF energy source connected to an ablation electrode of the catheter and electrically connected to the user interface through the patient interface unit. The ablation RF energy source is configured to produce an RF ablation signal to the ablation electrode in dependence upon a control signal received from the user interface. In an embodiment, the patient interface unit includes at least one low-pass filter configured to receive the electrical signals from the catheter.

In an embodiment, the patient interface unit includes a cross-point switch matrix electrically connected to the at least one low-pass filter, an analog-to-digital converter electrically connected to the switch matrix, and a digital interface circuit electrically connected to the analog-to-digital converter. In an embodiment, the patient interface unit is a high gain amplifier. In an embodiment, the at least one display includes a real-time display configured to display the electrical signals detected at the nerve site during or after the delivery of therapy to the nerve site, and a review display configured to display the electrical signals detected at the nerve site prior to the delivery of therapy. In an embodiment, the nerve site is within a renal artery of the patient.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope.

While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format and are not intended to be interpreted based on 35 U.S.C. § 122, sixth paragraph, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description or shown in the accompanying drawings shall be interpreted merely as examples illustrating the inventive concept herein and shall not be construed as limiting the invention.

What is claimed is:

1. A method for nervous system modulation, comprising the steps of:
operatively connecting an ECG cable having a plurality of surface electrodes to a body of a patient;
introducing a catheter having a plurality of catheter electrodes into a blood vessel of the patient;
probing a target location within the patient with the catheter to identify nerve tissue with maximum signal propagation in real-time; and
reducing signal propagation in the identified nerve tissue.

2. The method according to claim 1, wherein:
the step of reducing signal propagation in the identified nerve tissue includes ablating the identified nerve tissue via an ablation electrode of the catheter.

3. The method according to claim 2, wherein:
the ablation electrode is connected to a radiofrequency energy source.

4. The method according to claim 3, further comprising the step of:
monitoring the signal propagation in the nerve tissue while ablating the identified nerve tissue.

5. The method according to claim 4, further comprising the steps of:
monitoring the signal propagation in the nerve tissue at the target location after ablating the identified nerve tissue; and
comparing the signal propagation in the nerve tissue after ablating the identified nerve tissue, with the signal propagation in the identified nerve tissue before ablating the identified nerve tissue to determine if the modulation was successful.

6. The method according to claim 1, further comprising the step of:
steering the catheter to the target location utilizing at least one of fluoroscopy and electrical exploratory mapping.

7. The method according to claim 3, wherein:
the step of probing the target location to identify the nerve tissue with maximum signal propagation includes, at a patient interface unit, receiving electrical signals from the catheter electrodes of the catheter and the surface electrodes of the ECG cable and, at a user interface, displaying the signals on a display.

8. The method according to claim 7, wherein:
the display includes a real-time display and a review display.

9. The method according to claim 7, wherein:
ablating the identified nerve tissue includes, via the user interface, controlling an output signal from the radiofrequency energy source to the ablation electrode of the catheter.

10. The method according to claim 1, wherein:
the target location is within a renal artery of the patient;
the identified nerve tissue includes at least one renal nerve.

11. The method according to claim 1, wherein:
the step of reducing signal propagation in the identified nerve tissue includes ablating the identified nerve tissue via one of cryoablation and laser ablation.

12. A method of modulating the nervous system of a patient, comprising the steps of:
identifying a target location within the patient for exploration;
navigating a catheter to the target location;
detecting signal propagation within the nervous system at the target location, the signal propagation being indicative of nerve activity;
locating nerve tissue at the target location with maximum signal propagation;
ablating the nerve tissue with radiofrequency energy to reduce the signal propagation in the nerve tissue; and
during the step of ablating, monitoring the signal propagation in the nerve tissue.

13. The method according to claim 13, further comprising the step of:
storing signal propagation data representing the signal propagation in the nerve tissue prior to ablation; and
monitoring the signal propagation in the nerve tissue after ablating the nerve tissue.

14. The method according to claim 13, further comprising the step of:
comparing the signal propagation in the nerve tissue after ablating the nerve tissue with the stored signal propagation data to determine if the ablation was successful.

15. The method according to claim 12, wherein:
the step of navigating the catheter to the target location includes steering the catheter utilizing at least one of fluoroscopy and electrical exploratory mapping.

16. The method according to claim 12, wherein:
ablating the nerve tissue includes controlling the amount of the radiofrequency energy provided to an ablation electrode on a distal tip of the catheter.

17. The method according to claim 12, wherein:
the target location is within a renal artery of the patient;
the nerve tissue includes at least one renal nerve.

18. A system for nervous system modulation, comprising:
a user interface having at least one display associated therewith;
a patient interface unit operatively connected to the user interface, the patient interface unit being configured to receive electrical signals from electrodes of a catheter positioned at a nerve site within the body of a patient and surface electrodes of an ECG cable attached to the body of the patient, and to provide a digital output corresponding to the electrical signals to the user interface;
wherein the user interface is configured to display the electrical signals on the at least one display in real-time; and
wherein the user interface is configured to control the delivery of therapy to the nerve site to reduce signal propagation at the nerve site in dependence upon the real-time display of the signals.

19. The system of claim 18, further comprising:
an ablation RF energy source connected to an ablation electrode of the catheter and electrically connected to the user interface through the patient interface unit;
wherein the ablation RF energy source is configured to produce an RF ablation signal to the ablation electrode in dependence upon a control signal received from the user interface.

20. The system of claim 18, wherein:
the patient interface unit includes at least one low-pass filter configured to receive the electrical signals from the catheter.

21. The system of claim 20, wherein:
the patient interface unit includes a cross-point switch matrix electrically connected to the at least one low-pass filter, an analog-to-digital converter electrically connected to the switch matrix, and a digital interface circuit electrically connected to the analog-to-digital converter.

22. The system of claim 21, wherein:
the patient interface unit is a high gain amplifier.

23. The system of claim 18, wherein:
the at least one display includes a real-time display configured to display the electrical signals detected at the nerve site during or after the delivery of therapy to the nerve site, and a review display configured to display the electrical signals detected at the nerve site prior to the delivery of therapy.

24. The system of claim 18, wherein:
the nerve site is within a renal artery of the patient.
